(19) United States
(12) Patent Application Publication
(10) Pub. No.: US 2007/0259968 A1
(43) Pub. Date: Nov. 8, 2007

(54) Macroporous Plastic Bead

(75) Inventors: Stefan Menzler, Darmstadt (DE); Christian Meier, Darmstadt (DE); Roger Recktenwald, Bensheim (DE); Sabine Schwarz-Barac, Riedstadt-Wolfskehln (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GMBH, Darmstadt (DE)

(21) Appl. No.: 11/719,969

(22) PCT Filed: Nov. 8, 2005
(86) PCT No.: PCT/EP05/11907

§ 371 Date: May 23, 2007

(30) Foreign Application Priority Data
Feb. 8, 2005 (DE) 10 2005 005 828.0

(51) Int. Cl. A61K 47/32; B01D 15/08; C08F 110/00

(52) U.S. Cl. 514/772.4; 210/656; 521/142

(57) ABSTRACT

(EN) The invention relates to a macroporous plastic bead material with a mean particle diameter of 10 to 1000 pm, radically polymerised from the following monomer types a) 5-40 wt. % vinylic polymerisable monomers with a water-solubility of at least 1% at 20° C. which are not vinylic polymerisable monomers with a quaternary amino group, b) 5-50 wt. % of vinylic polymerisable monomers with an additional functional group which can for covalent bonds on reaction with nucleophilic groups of ligands, c) 20-60 wt. % hydrophilic, cross-linking radically polymerisable monomers with two or more ethylenically-unsaturated polymerisable groups, characterised in that in addition d) 1 to 20 wt. % of a vinylic polymerisable monomer with a quaternary amino group are used for the polymer. The invention further relates to a method for production of the macroporous plastic bead material by inverse suspension polymerisation of monomer phase and uses thereof.

The invention relates to a macroporous synthetic polymer bead material. The synthetic polymer bead material is a crosslinked copolymer which is composed of hydrophilic monomers capable of vinylic polymerization and which has binding activity with respect to ligands having nucleophilic groups. The invention further relates to a process for preparation of the macroporous synthetic polymer bead material via inverse suspension polymerization of a monomer phase, and also to uses of the material.

### PRIOR ART

Porous polymeric carrier materials for proteins, in particular enzymes, are well known. Fields of application lie within the medical sector, e.g. in the enzymatic cleavage of β-lactam antibiotics, such as penicillin G, to give 6-aminopenicillinic acid (6-APA) by means of penicillin acylase (penicillinamidase). Especially important development aims are maximum loading capacity, but also low swellability and minimum residual solvent contents. Halogenated solvents are fundamentally to be avoided during the preparation process.

DE-A 22 37 316 describes a process for preparation of crosslinked bead copolymers via free-radical polymerization of a monomer mixture which comprises a free-radical-generating initiator and which comprises a monomer which has binding activity with respect to biological substances, and comprises a crosslinking comonomer and at least one other comonomer, the monomer mixture being suspended in a non-polar organic liquid to give droplets, and polymerized. Suitable non-polar organic liquids are in particular aliphatic hydrocarbons, especially those having 6 or more carbon atoms. In the Examples, mixtures composed of n-heptane and perchloroethylene are used. The ratio of the monomer phase to the continuous organic phase can be from 1:1 to 1:10, but preferred ratios are from 1:1.5 to 1:4.

DE-A 31 06 456 describes a process improved with respect to DE-A 22 37 316 in relation to the binding capacity of the polymer beads. Particularly high binding capacities for proteins, in particular for the enzyme penicillin acylase (penicillin amidase) are obtained if the carrier polymers have high contents of crosslinking monomers, and if the monomer phase, formed from the monomers and from the diluent, comprises a solvent mixture as diluent. Examples of suitable mixtures can be water/methanol or formamide/methanol. Monomers and diluent are present for example in a ratio of 1:2.6. For the organic, continuous phase, a mixture composed of n-hexane and perchloroethylene is used. The ratio of the monomer phase to the continuous organic phase is about 1:2.8 in the Examples. At crosslinking agent contents of 50% by weight in the monomer mixture, and using water/methanol as diluent it is possible to obtain carrier polymers whose binding capacity is up to 125 U/g, measured as penicillin acylase activity.

DE 34 04 021 A1 describes macroporous bead polymers in which, unlike in the present invention, epoxy groups have been introduced subsequently. The loading described inter alia with penicillin acylase is comparable with the process of the present application. Relatively high binding capacities are achieved on the material moist from suction filtration. If the value likewise stated in each case and based on the dry weight is considered, the swelling index can be calculated indirectly (U/g moist U/g dry). The values thus obtained are in the region of about 3.0.

DE 198 04 518 describes a process for preparation of bead copolymers based on acrylate, carrier polymer materials prepared accordingly, and their uses. A feature, inter alia, of the carrier polymer material is at least 220 [U/g, moist] binding capacity for penicillin amidase and at the same time a low swelling index of at most 1.5.

EP 1 352 95, A1 describes carrier materials having binding activity and comprising epoxy groups for the immobilization of enzymes. An advantage of the carrier material described is that enzymes can be bound covalently even at low ionic strengths. The functionality is achieved in a process in which, using carrier bead materials having epoxy groups at the surface, some of the epoxy groups are subsequently reacted with various reagents. The result of this is additional amino groups, which promote the binding of the enzymes at low ionic strengths in the ambient medium.

### OBJECT AND ACHIEVEMENT OF OBJECT

DE 198 04 518 C2 describes a process for preparation of bead copolymers based on acrylate and carrier polymer materials prepared accordingly having excellent properties in particular high binding capacity for penicillin amidase and at the same time low swelling index. A disadvantage of these carrier polymer materials is seen in the fact that covalent bonding of biomacromolecules has to take place at comparatively high ionic strength.

The disadvantages consist inter alga in that the wastewater produced in the process pollutes the environment because of the high salt content. A high salt content can moreover cause some damage to or denaturing of the biomacromolecules to be immobilized.

EP 1 352 957 A1 proposes a solution to this problem wherein, starting from carrier bead materials having epoxy groups at the surface, some of the epoxy groups are subsequently reacted with various reagents. This produces additional amino groups, which promote the binding of the enzymes at low ionic strengths in the ambient medium. This process has the disadvantage of being complicated and therefore increasing the cost of preparation of the carrier polymer materials.

Taking DE 198 04 518 as a starting point, the intention was to provide a macroporous synthetic polymer bead material which permits covalent binding of biomacromolecules at comparatively low ionic strength. The intention here was to avoid complicated subsequent modification as described in EP 1 352 957 A1. At the same time, the intention is that the swelling index remain within the range of acceptable values being not higher than 2.5.

The object is achieved via a

macroporous synthetic polymer bead material whose average particle diameter is from 10 to 1000 μm, free-radical-polymerized from the following types of monomer

a) from 5 to 40% by weight of monomers capable of vinylic polymerization whose solubility in water is at least 1% at 20° C., other than monomers capable of vinylic polymerization and having a quaternary amino group,
b) from 5 to 50% by weight of monomers capable of vinylic polymerization having an additional functional group which can enter into a reaction with nucleophilic groups of ligands to give covalent bonds,
c) from 20 to 60% by weight of hydrophilic cross-linking monomers capable of free-radical polymerization having two or more ethylenically unsaturated polymerizable groups,
   characterized in that the polymer also uses
d) from 1 to 20% by weight of a monomer capable of vinylic polymerization having a quaternary amino group.
   Exposition of the Invention
   Monomers

In order to ensure that the monomer mixture is hydrophilic, it must be composed mainly of hydrophilic monomers. Hydrophilic monomers are those monomers which form aqueous solutions of strength at least 10% at room temperature, and preferably comprise no ionic groups or groups ionizable via addition of acid or of base.

The entirety of the monomers a), b), c) and d) always gives a total of 100% by weight.

Monomers a)

The monomers a) are from 5 to 40%, preferably from 5 to 20% and in particular from 6 to 10% by weight of hydrophilic monomers capable of free-radical polymerization and having a vinyl group, which form aqueous solutions of strength at least 10% at room temperature. Monomers a) are not monomers capable of vinylic polymerization and having a quaternary amino group. The monomers a) therefore always differ from the monomers d).

Particularly suitable monomers a) are acrylamide and/or methacrylamide, methacrylamide being preferred. Other examples are hydroxyalkyl esters of unsaturated polymerizable carboxylic acids, e.g. hydroxyethyl acrylate and hydroxyethyl methacrylate, or N-vinylpyrrolidone.

Monomers b)

Monomers b) are from 5 to 50% by weight, preferably from 32 to 40% by weight, or monomers capable of free-radical polymerization and having a vinyl group and having an additional functional group, preferably an oxirane group (epoxy group) which can enter into a polymer-analogous reaction with the nucleophilic groups of the ligands to form covalent bonds. In particular, oxirane groups are suitable for binding ligands with retention of their biological activity.

Preferred monomers b) are glycidyl methacrylate and/or allyl glycidyl ether. It is particularly preferable to use both monomers simultaneously in approximately equal amounts so that together their proportion is from 30 to 50% by weight, preferably from 32 to 40% by weight.

Monomer c)

Monomers c) are from 20 to 60% by weight, in particular from 25 to 55% by weight, particularly preferably from 40 to 55% by weight, of hydrophilic, crosslinking monomers capable of free-radical polymerization and having two or more ethylenically unsaturated polymerizable groups. Preferred monomers c) are N,N′-methylenebisacrylamide or N,N′-methylenbismethacrylamide. N,N′-Methylenebismethacrylamide is particularly preferred. It is also possible to use, if appropriate, from 0 to 10% by weight of other crosslinking monomers capable of free-radical polymerization and having two or more ethylenically unsaturated polymerizable groups. Hydrophilic di(meth)-acrylates are suitable, e.g. polyethylene oxide di(meth) acrylates.

Monomers d)

Monomers d) are from 1 to 20% by weight, preferably from 5 to 15% by weight, in particular from 8 to 12% by weight, of monomers capable of vinylic polymerization and having a quaternary amino group, preferably alkyl (meth)acrylate monomers having a quaternary amino group in the alkyl radical. Preferred monomers d) are trimethylammoniumethyl methacrylate or trimethylammoniumethyl methacrylate chloride.

Preferred Monomer Compositions

The macroporous synthetic polymer bead material is preferably a copolymer composed of the following monomers:
a) acrylamide and/or methacrylamide
b) glycidyl methacrylate and/or allyl glycidyl ether
c) N,N′-methylenebisacrylamide or N,N′-methylenebismethacrylamide
d) trimethylammoniumethyl methacrylate or to be more precise trimethylammoniumethyl methacrylate chloride.

One particularly preferred composition, where the proportions of the five monomers mentioned of monomer types a), b), c) and d) give a total of 100% by weight is:
a) from 6 to 10% by weight of methacrylamide
b) from 16 to 20% by weight of glycidyl methacrylate and from 16 to 20% by weight of allyl glycidyl ether
c) from 46 to 50% by weight of N,N′-methylenebismethacrylamide
d) from 8 to 12% by weight of trimethylammoniumethyl methacrylate chloride
   Process for Preparation of the Copolymer

The process substantially corresponds to that of DE 198 04 518 C2, with the proviso that the monomer d) is an essential constituent of the monomer mixture.

The invention therefore provides a process for preparation of a crosslinked hydrophilic bead copolymer having activity with respect to binding of ligands having nucleophilic groups, via conventional inverse bead polymerization of a monomer phase composed of monomers and of a diluent, where the monomers present for the copolymer comprise
a) from 5 to 40% by weight of hydrophilic monomers capable of free-radical polymerization having a vinyl group which at room temperature form at least 10% strength aqueous solutions, other than monomers capable of vinylic polymerization and having a quaternary amino group,
b) from 5 to 50% by weight of monomers capable of free-radical polymerizaiton having a vinyl group and having an additional functional group which can enter into a polymer-analogous reaction with the nucleophilic groups of the ligands to give covalent bonds, and
c) from 20 to 60% by weight of hydrophilic crosslinking monomers capable of free-radical polymerization having two or more ethylenically unsaturated polymerizable groups, and also
d) from 1 to 20% by weight of a vinylically polymerizable monomer having a quaternary amino group,
   with the proviso that a), b), c) and d) give a total of 100% by weight and the ratio of the monomers to the diluent is from 1:1.5 to 1:2.5, preferably from 1:1.7 to 1:2.3, and the diluent used comprises a mixture composed of methanol and water in a ratio of from 1:1.0 to 1:4.0, where the monomer phase has been dispersed in a continuous phase composed of an organic solvent composed of an aliphatic hydrocarbon having from 5 to 7 carbon atoms, to give droplets, and where the ratio of monomer phase to continuous phase is from 1:1.5 to 1:4.0 preferably from 1:2.0 to 1:3.0, and the monomers in this form undergo free-radical polymerization in the presence of a polymerization initiator and of a protective colloid.
   Diluent

The monomer phase is composed of the monomers a), b), c) and d), dissolved in a diluent which has to be a mixture composed of methanol and water in a ratio of from 1:1.0 to 1:4.0. Particularly advantageous mixing ratios for methanol and water are from 1:1.2 to 1:2.5, in particular from 1:1.3 to 1:1.7

Ratio of Monomers to Diluent

The ratio of monomers to diluent is particularly critical. This has to be in the range from 1:1.5 to 1:2.5, preferably from 1:1.7 to 1:2.3 particularly preferably in the range from 1.9 to 2.1.

Continuous Phase

A suitable continuous phase is an organic solvent which is an aliphatic hydrocarbon having from 4 to 7 carbon atoms. n-Heptane is preferred and cyclohexane is particularly preferred.

Ratio of Monomer Phase/Continuous Phase

The ratio of the monomer phase to the continuous phase formed by the organic solvent has to be from 1:1.5 to 1:4.0, preferably from 1:2.0 to 1:3.0.

Other Process Conditions

Other constituents present in the suspended monomer phase are, in a manner known per se, polymerization initiators, preference being given to sulphur-free initiators, and particular preference being given to 4,4′-azobis-(4-valeric acid) and protective colloids (emulsifiers), e.g. a copolymer composed of 95 parts of n-butyl methacrylate and of 5 parts of 2-trimethylammoniumethyl methacrylate chloride with molecular weights (weight-average) in the range from 30 000 to 80 000.

The bead polymerization process (also termed suspension polymerization) is in other respects conducted in a known manner, for example by using the continuous phase with the protective colloid as initial charge and distributing the monomer phase, which also includes the initiators with stirring, for example at from 40 to 60° C. in the organic phase, and then heating to 60-70° C. The water/methanol mixture can, for example, be almost completely separated by an azeotropic method over a period of 6 hours. The mixture is permitted to react to completion for about 3-5 hours and is then cooled to room temperature. The resultant beads are isolated by suction filtration and, for example, dried in vacuo for 12 hours. As an alternative to this, the bead polymers car also be filtered off and washed with water and then used in water-moist form, or dried. The drying is preferably undertaken in a fluidized-bed dryer, because this method is particularly effective in removing solvent residues. The size of the resultant polymer beads (=carrier polymer material) is in the range from 50 to 500 μm, in particular from 120 to 250 μm.

Binding Capacity

An important field of application for the inventive carrier polymer material is the cleavage of penicillin G to give 6-aminopenicillinic acid (6-APA) by means of bound penicillin amidase derived from E. coli.

The binding capacity is that enzymatic activity which can be achieved using maximum loading of the carrier polymer material with a certain enzyme. The binding capacity is expressed as penicillin amidase activity in units per g of carrier polymer beads [U/g, moist]. The binding capacity of the inventive carrier polymer beads is at least 200 [U/g, moist], using this measurement method.

The binding capacity of the inventive macroporous synthetic polymer bead material for penicillin amidase derived from E. coli is at least 200 [U/g, moist], resulting from the reaction of 1530 units of penicillin amidase with 1 g of carrier polymer material, in the presence of a salt concentration of at most 0.1, preferably at most 0.05 [mol/l]. The salt concentration is determined by calculation from any salt present in the enzyme solution and the salt added for immobilization or the buffer salt in the immobilizing mixture.

Methods

Determination of binding capacity for penicillin amidase at various salt concentrations

Determination of Binding Capacity for Penicillin Amidase (=Penicillin G Acylase) Derived from E. coli (EC 3.5.1.11)

a) Covalent binding of penicillin amidase to the carrier polymer material

1 g of carrier polymer material is added to 1530 units of penicillin amidase in 5 ml of sterile potassium phosphate buffer, pH value 7.5, and incubated at 23° C. for 48 hours.

The polymer beads are then placed on a frit composed of sintered glass (porosity 2 or 3) and washed twice with deionized water and then twice with 0.1 M potassium phosphate buffer, pH 7.5, comprising 0.05% of ethyl 4-hydroxybenzoate, by means of suction filtration on the frit. The moist weight of the resultant beads loaded with penicillin acylase is determined.

b) Determination of binding capacity

400-700 mg of moist penicillin-amidase-coupled carrier polymer material (polymer beads) are placed in 30 ml of a 2% strength penicillin G solution in 0.05 M potassium phosphate buffer, pH 7.5, comprising 0.05% of ethyl 4-hydroxybenzoate, at 37° C.

Liberated phenyl acetic acid is titrated with 0.5 M NaOH with uniform stirring at a constant pH of 7.8 for a period of 4 minutes, recording the consumption of NaOH.

The polymer beads are then obtained as in a) by way of a glass frit by using suction to pass 20 ml of deionized water through the material.

c) Calculation of binding capacity

The calculation is based on the linear region of the measurement curves (usually the region from 1 to 3 min). The binding capacity is stated as penicillin amidase units per g of moist carrier polymer material (U/g, moist). One unit corresponds to one μmol of hydrolysed penicillin G per minute (μmol/min);

1 l of 0.5M NaOH here is equivalent to 500 μmol of hydrolysed penicillin G. (The water content of the carrier polymer material is approximately constant and can therefore be ignored.)

Determination of Penicillin Amidase Binding Yield in [%]

The penicillin amidase (PcA) binding yield is calculated from the formula
PcA binding yield [%]=A[U/g]×F[g]×100/PcA[U]

| | |
|---|---|
| A = | immobilized activity, PcA/g of moist immobilizate |
| F = | Moist yield = moist weight of 1 g of dry polymer carrier |
| PcA = | units of PcA used per g of dry polymer carrier |

Swelling Index

The swellability of the polymer beads in water is expressed via the swelling index [ml, moist/ml, dry]. The swelling index of the inventive macroporous synthetic polymer bead material in water is greater than 1.5-2.5, preferably 1.7-2.3.

The swelling index is therefore higher than for the synthetic polymer bead material according to DE 198 04 518 C2 (<1.5) and lower than for the synthetic polymer bead material according to DE 34 04 021 A1 (about 3.0).

Determination of Swelling Index [ml, moist/ml dry]

1 g of dry polymer carrier is weighed into a 25 ml measuring cylinder. The fill height in ml is determined (=dry volume). The measuring cylinder is then ⅔-filled with 0.01% aqueous polysorbate 80 solution. The measuring cylinder is shaken 6 times, at intervals of 10 min. Beads adhering to the wall are returned by flushing with 5 ml of aqueous polysorbate 80 solution. After 3 h, the moist volume of the moist polymer carrier on the base of the measuring cylinder is read off in ml. The quotient calculated for moist volume/dry volume gives the swelling index.

Uses

The inventive carrier polymer materials can be used for covalent binding of ligands by means of the oxirane groups present, in stirred reactors or -low reactors. This can be achieved for example, via attachment of proteins in particular of enzymes, from concentrated solutions by way of covalent bonding with retention of their biological activity. It is moreover also possible to react peptides, amino acids, β-lactam antibiotics, lipids, nucleotides, polynucleotides, low-molecular-weight nucleophilic compounds or organometallic compounds with the oxirane groups of the carrier beads.

The polymer beads loaded with liigands can be used in a manner known per se for the stereospecific synthesis of chiral substances, such as amino acids (D-phenylalanine, p-hydroxy-D-phenylalanine, L-tert-leucine), or of medicaments, e.g. of ibuprofen. They are also used as carriers in the enzymatic cleavage of penicillin G to give 6-aminopenicillinic acid (6-APA), cephalosporin G to give 7-aminodesacetoxycephalosporanic acid (7-ADCA) or cephalosporin C to give 7-aminocephalosporanic acid (7-ACA). The process is described in DECHEMA Jahrestagung 1996—Kurzfassungen [Dechema annual conference 1996—abstracts], Vol. 1, DECHEMA e.V. Other fields of application are specific enzymatic syntheses on substrates, e.g. above cleavage products to give amoxicillin and ampicillin. Another field of application is syntheses of fine chemicals or of starting materials for chemical syntheses (e.g. malic acid, malate). Other uses are the hydrolysis of lactose with carrier-fixed β-galactosidase and the decomposition of hydrogen peroxide using carrier-fixed catalase. The polymer beads can also be used in separation technology, for adsorption chromatography or gel permeation chromatography. For specific adsorption, the polymer beads can be loaded with immunoglobulin fractions derived from antisera or with monoclonal antibodies. Another field of application is the use of the carrier polymer material loaded with enzymes or with antibodies as adsorbant in extracorporeal therapy, in which pathogenic or toxic substances are removed from whole blood.

The inventive macroporous synthetic polymer bead material can in particular be used:
For the binding of proteins.
In chromatography.
For the synthesis of medicaments.

For stereospecific synthesis to obtain enantiomerically pure substances.
For the binding of enzymes.
For the binding of antibodies.
   Advantageous Properties of the Invention

The monomer present in the inventive macroporous synthetic polymer bead material and capable of vinylic polymerization and having a quaternary amino group permits the physical adsorption of ligands, e.g. enzymes, by way of ionic interactions irrespective of the pH of the immobilization mixture. In contrast to EP 1 352 957 A1, it is possible to prepare the synthetic polymer bead material in one step without post-treatment operations on the polymer. However, ligands can be bound covalently in a manner similar to that for the EP 1 352 957 A1 material, even at extremely low salt content, with good yield. Despite introduction of the hydrophilic monomers capable of vinylic polymerization and having a quaternary amino group, the swelling index of the synthetic polymer bead material is within acceptable ranges.

### EXAMPLES

### Methods

Determination of Binding Capacity for Penicillin Amidase at Various Salt Concentrations

Determination of Binding Capacity for Penicillin Amidase (=Penicillin G Acylase) Derived from E. coli (EC 3.5.1.11)

a) Covalent binding of penicillin amidase to the carrier polymer material

1 g of carrier polymer material is added to 1530 units of penicillin amidase in 5 ml of sterile potassium phosphate buffer, pH value 7.5, and incubated at 23° C. for 48 hours.

The polymer beads are then placed on a frit composed of sintered glass (porosity 2 or 3) and washed twice with deionized water and then twice with 0.1 M potassium phosphate buffer, pH 7.5, comprising 0.05% of ethyl 4-hydroxybenzoate, by means of suction filtration on the frit. The moist weight of the resultant beads loaded with penicillin acylase is determined.

b) Determination of binding capacity

400-700 mg of moist penicillin-amidase-coupled carrier polymer material (polymer beads) are placed in 30 ml of a 2% strength penicillin G solution in 0.05 M potassium phosphate buffer, pH 7.5, comprising 0.05% of ethyl 4-hydroxybenzoate, at 37° C.

Liberated phenyl acetic acid is titrated with 0.5 M NaOH with uniform stirring at a constant pH of 7.8 for a period of 4 minutes, recording the consumption of NaOH.

The polymer beads are then obtained as in a) by way of a glass frit by using suction to pass 20 ml of deionized water through the material.

c) Calculation of binding capacity

The calculation is based on the linear region of the measurement curves (usually the region from 1 to 3 min). The binding capacity is stated as penicillin amidase units per g of moist carrier polymer material (U/g, moist). One unit corresponds to one μmol of hydrolysed penicillin G per minute (μmol/min);

1 l of 0.5M NaOH here is equivalent to 500 μmol of hydrolysed penicillin G. (The water content of the carrier polymer material is approximately constant and can therefore be ignored.)

Determination of Penicillin Amidase Binding Yield in [%]

The penicillin amidase (PcA) binding yield is calculated from the formula
PcA binding yield [%]=A[U/g]×F[g]×100/PcA[U]

| | |
|---|---|
| A = | immobilized activity, PcA/g of moist immobilizate |
| F = | moist yield = moist weight of 1 g of dry polymer carrier |
| PcA = | units of PcA used per g of dry polymer carrier |

Determination of Swelling Index [ml, moist/ml, dry]

1 g of dry polymer carrier is weighed into a 25 ml measuring cylinder. The fill height in ml is determined (=dry volume). The measuring cylinder is then ⅔-filled with 0.01% aqueous polysorbate 80 solution. The measuring cylinder is shaken 6 times, at intervals of 10 min. Beads adhering to the wall are returned by flushing with 5 ml of aqueous polysorbate 80 solution. After 3 h, the moist volume of the moist polymer carrier on the base of the measuring cylinder is read off in ml. The quotient calculated for moist volume/dry volume gives the swelling index.

### EXAMPLES 1-3

### Identical Experimental Conditions in Examples 1-3

An organic solvent and 3 g of a copolymer composed of 95 parts by n-butyl methacrylate and of 5 parts of 2-trimethylammoniumethyl methacrylate chloride as protective colloid are used as initial charge in a 2 l stirred flask with thermometer, reflux condenser, nitrogen inlet tube. A monomer phase composed of diluent, and also 100 g of the monomer mixture stated in Table 1

and also

2 g of 4,4′-azobis-4-cyanovaleric acid (as polymerization initiator)

are dispersed at 50° C., with stirring and nitrogen flushing, in the organic phase, and the mixture is then heated to boiling at from 65 to 70° C. The mixture is stirred for about 6 hours and then cooled to room temperature. The resultant polymer beads are isolated by suction filtration, washed, and dried in a fluidized-bed dryer. The binding capacity for penicillin amidase [U/g, moist] is then determined at various salt concentrations, and the binding yield and the swelling index [ml, moist/ml, dry] are determined. The results are collated in Table 2.

TABLE 1

| | | Example 2 | Example 3 |
|---|---|---|---|
| | Example 1 | (Comparative | (Comparative |
| | (Inventive) | example) | example) |
| Organic solvent | 500 g of | 952 g of | 669 g of |
| (continuous phase) | cyclohexane | cyclohexane | cyclohexane |
| Monomers [g] | | | |
| Methacrylamide | 8 | 10 | 10 |
| Allylglycidyl ether | 18 | 20 | 20 |
| Glycidyl | 18 | 20 | 20 |
| methacrylate | | | |
| Methylene- | 48 | 50 | 50 |
| bismethacrylamide | | | |
| Trimethylammonium- | 10 | — | — |
| ethyl methacrylate | | | |
| chloride | | | |
| Diluent | 80 g of | 80 g of | 263 g of |
| | methanol + | methanol + | formamide |
| | 120 g of | 120 g of | |
| | water (=1:1.5) | water (=1:1.5) | |
| Monomers + | 300 g | 300 g | 363 g |
| diluent (monomer | | | |
| phase) | | | |
| Monomers/diluent | 1:2 | 1:2 | 1:2.63 |
| ratio | | | |
| Monomer | 1:2.0 | 1:3.2 | 1:1.8 |
| phase/continuous | | | |
| phase ratio | | | |

TABLE 2

| | Binding capacity for penicillin amidase | | |
|---|---|---|---|
| | (1530 U) [U/g, moist] at [mol/l] | | |
| | salt concentration/yield [%] | | |
| | | Example 2 | Example 3 |
| | Example 1 | (Comparative | (Comparative |
| | (Inventive) | example) | example) |
| 0 | 202/49 | 96/19 | 220/56 |
| 0.2 | 207/49 | 153/30 | 238/59 |
| 0.4 | 236/68 | 192/38 | 250/62 |
| 0.6 | 240/60 | 216/42 | 252/63 |
| 0.8 | 229/55 | 208/41 | 222/55 |
| 1.0 | 245/61 | 235/46 | 215/52 |
| Swelling index | 1.7 | 1.3 | 4.0 |
| [ml, moist/ml, dry] | | | |

1. A macroporous synthetic polymer bead material having an average particle diameter from 10 to 1000 μm, wherein the macroporous synthetic polymer bead material is free-radical-polymerized from the following types of monomer
a) from 5 to 40% by weight of monomers capable of vinylic polymerization whose solubility in water is at least 1% at 20° C., other than monomers capable of vinylic polymerization and having a quaternary amino group,
b) from 5 to 50% by weight of monomers capable of vinylic polymerization having an additional functional group which can enter into a reaction with nucleophilic groups of ligands to give covalent bonds.
c) from 20 to 60% by weight of hydrophilic, crosslinking monomers capable of free-radical polymerization having two or more ethylenically unsaturated polymerizable groups,
wherein the macroporous synthetic polymer bead material further comprises
d) from 1 to 20% by weight of a monomer capable of vinylic polymerization having a quaternary amino group.

2. The macroporous synthetic polymer bead material according to claim 1, wherein monomer d) is an alkyl (meth)acrylate having a quaternary amino group in the alkyl radical

3. The macroporous synthetic polymer bead material according to claim 1, wherein the macroporous synthetic polymer bead material has a swelling index in water of greater than 1.5-2.5 and has a binding capacity to penicillin amidase derived from E. coli of at least 200 U/g, moist resulting from the reaction of 1530 units of penicillin amidase with 1 g of carrier polymer material, in the presence of a salt concentration of at most 0.1 mol/l.

4. The macroporous synthetic polymer bead material according to claim 1, wherein the macroporous synthetic polymer bead material is a copolymer composed of the following monomers:
a) acrylamide and/or methacrylamide;
b) glycidyl methacrylate and/or allyl glycidyl ether;
c) N,N′-methylenebisacrylamide or N,N′-methylenebismethacrylamide; and
d) trimethylammoniumethyl methacrylate or trimethylammoniumethyl methacrylate chloride.

5. The macroporous synthetic polymer bead material according to claim 4, wherein the macroporous synthetic polymer bead material is a polymer composed of the following monomers, which when taken together give a total of 100% by weight:
a) from 6 to 10% by weight of methacrylamide;
b) from 16 to 20% by weight of glycidyl methacrylate and from 16 to 20% by weight of allyl glycidyl ether;
c) from 46 to 50% by weight of N,N′-methylenebismethacrylamide; and
d) from 8 to 12% by weight of trimethylammoniumethyl methacrylate chloride.

6. A process for preparing a crosslinked hydrophilic bead copolymer having activity with respect to binding of ligands having nucleophilic groups, via inverse bead polymerization of a monomer phase composed of monomers and of a diluent, wherein the monomers comprise
a) from 5 to 40% by weight of hydrophilic monomers capable of free-radical polymerization having a vinyl group which at room temperature form at least 10% strength aqueous solutions other than monomers capable of vinylic polymerization and having a quaternary amino group,
b) from 5 to 50% by weight of monomers capable of free-radical polymerization having a vinyl group and having an additional functional group which can enter into a polymer-analogous reaction with the nucleophilic groups of the ligands to give covalent bonds,
c) from 20 to 60% by weight of hydrophilic crosslinking monomers capable of free-radical polymerization having two or more ethylenically unsaturated polymerizable groups, and
d) from 1 to 20% by weight of an alkyl methacrylate monomer having a quaternary amino group in the alkyl radical,
with the proviso that a), b), c) and d) give a total of 100% by weight, and the ratio of the monomers to the diluent is from 1:1.5 to 1:2.5, and the diluent used comprises a mixture composed of methanol and water in a ratio of from 1:1.0 to 1:4.0, where the monomer phase has been dispersed in a continuous phase composed of an organic solvent composed of an aliphatic hydrocarbon having on 5 to 7 carbon atoms, to give droplets, and where the ratio of monomer phase to continuous phase is on 1:1.5 to 1:4.0 and the monomers in this form undergo free-radical polymerization in the presence of a polymerization initiator and of a protective colloid.

7. The process according to claim 6, wherein the monomers comprise
a) acrylamide and/or methacrylamide,
b) glycidyl methacrylate and/or allyl glycidyl ether,
c) N,N′-methylenebisacrylamide or N,N′-methylenebismethacrylamide, and
d) trimethylammoniumethyl methacrylate chloride.

8. The process according to claim 6, wherein the organic solvent is cyclohexane.

9-14. (canceled)

15. A method of binding a protein comprising adding to the protein a carrier comprising the macroporous synthetic polymer bead material according to claim 1.

16. A method of separating a protein comprising adding the protein to a chromatograph comprising the macroporous synthetic polymer bead material according to claim 1.

17. A method of synthesizing a medicinal substance comprising enzymatically cleaving a substrate in the presence of a carrier comprising the macroporous synthetic polymer bead material according to claim 1.

18. A method of isolating an enantiomerically pure substance comprising adding to an enantiomeric mixture of a substance a carrier comprising the macroporous synthetic polymer bead material according to claim 1.

19. A method of binding an enzyme comprising adding to the enzyme a carrier comprising the macroporous synthetic polymer bead material according to claim 1.

20. A method of binding ana antibody comprising adding to the antibody a carrier comprising the macroporous synthetic polymer bead material according to claim 1.

21. The macroporous synthetic polymer bead material according to claim 2, wherein the alkyl (meth)acrylate, which has a quaternary amino group in the alkyl radical, of monomer d) is selected from the group consisting of trimethylammoniumethyl methacrylate and trimethylammoniumethyl methacrylate chloride.
